(19) United States
(12) Patent Application Publication
(10) Pub. No.: US 2003/0054974 A1
(43) Pub. Date: Mar. 20, 2003

(54) Non-oxidatively metabolized compounds and compositions, synthetic pathways therefor, and uses thereof

(75) Inventor: Pascal Druzgala, Santa Rosa, CA (US)

Correspondence Address: SALIWANCHIK LLOYD & SALIWANCHIK, A PROFESSIONAL ASSOCIATION, 2421 N.W. 41ST STREET, SUITE A-1, GAINESVILLE, FL, 326066669

(73) Assignee: Aryx Therapeutics, Santa Clara, CA

(21) Appl. No.: 10/228,670

(22) Filed: Aug. 26, 2002

Related U.S. Application Data
(60) Provisional application No. 60/314,792, filed on Aug. 24, 2001.

(51) Int. Cl. A61K 31/00; C07C 27/00

(52) U.S. Cl. 514/1; 568/959

(57) ABSTRACT

The subject invention provides therapeutically useful and therapeutically effective compounds and compositions for the treatment of a variety of disorders. The compounds of the invention exhibit significantly reduced levels of drug-drug interactions (DDI) and are metabolized, primarily, via non-oxidative systems.

### CROSS-REFERENCE TO RELATED APPLICATION

The present application claims priority to U.S. Provisional Application Serial No. 60/314,792, filed Aug. 24, 2001, which is hereby incorporated by reference herein in its entirety, including any figures, tables, nucleic acid sequences, amino acid sequences, or drawings.

### BACKGROUND OF INVENTION

Adverse drug-drug interactions (DDI), elevation of liver function test (LFT) values, and QT prolongation leading to torsades de pointes (TDP) are three major reasons why drug candidates fail to obtain FDA approval. All these causes are, to some extent metabolism-based.

Oxidative metabolism is the primary metabolic pathway by which most drugs (xenobiotics) are eliminated. It is also the major source of drug toxicity, either intrinsic toxicity or toxicity due to drug-drug interactions (DDI). Adverse DDI as well as intrinsic toxicity due to metabolites are a major reason for the failure of drug candidates in late-stage clinical trials. Many DDI are metabolism based, i.e., two or more drugs compete for the same metabolizing enzyme in the cytochrome P450 system (CYP450) (Guengerich, F. P., “Role of cytochrome P450 enzymes in drug-drug interactions,” Drug-drug interactions: scientific and regulatory perspectives (1997) 7-35, Li AP (ed.) Academic Press, San Diego; Shen, W. W., “Cytochrome P450 monooxygenases and interactions of psychotropic drugs: a five-year update,” Int. J. Psychiatry Med. (1995) 25:277-290). Non-oxidative metabolic systems, such as hydrolytic enzymes, on the other hand, do not depend on co-factors; are not inducible; have a high substrate capacity; do not have a high degree of inter-individual variations in man; and are present in most tissues and organs. Non-oxidative metabolic systems are, therefore, much more reliable.

Metabolism-based DDI take place when two (2) or more drugs compete for metabolism by the same enzyme. These metabolic interactions become relevant to DDI when the metabolic system is inducible or/and easily saturable. Such metabolic interactions lead to modification of the pharmacokinetics of the drugs and potential toxicity.

Multiple-drug therapy is a common practice, particularly in patients with several diseases or conditions. Whenever two or more drugs are administered over similar or overlapping time periods, the possibility of drug interactions exists. The ability of a single CYP to metabolize multiple substrates is responsible for the large number of documented clinically significant drug interactions associated with CYP inhibition (Shen, W. W., “Cytochrome P450 monooxygenases and interactions of psychotropic drugs: a five-year update,” Int. J. Psychiatry Med. (1995) 25:277-290; Riesenman, C., “Antidepressant drug interactions and cytochrome P450 system: a critical appraisal,” Pharmacotherapy (1995) 15:84S-99S; Somogyi, A. et al., “Pharmacokinetic interactions of cimetidine,” Clin Pharmacokinet (1987) 12:321-366). The inhibition of drug metabolism by competition for the same enzyme may result in undesirable elevation in plasma drug concentration. In addition, drug interactions can also occur as a result of induction of several CYPs following prolonged drug treatment.

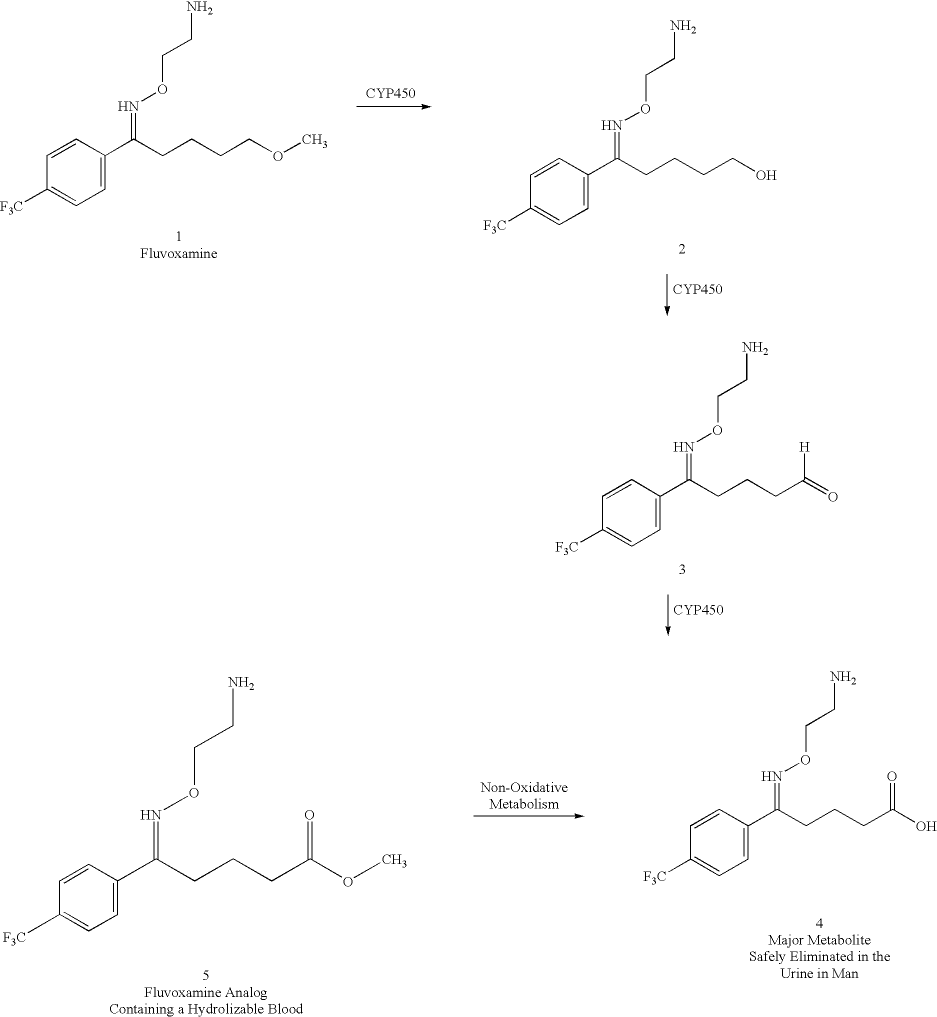

The non-oxidative metabolic concept of this invention is best explained by specific examples and is illustrated, above, in the case of fluvoxarnine (Luvox® 1). Fluvoxamine is a serotonin reuptake inhibitor that is useful in the treatment of certain compulsive disorders in man. Fluvoxamine was developed at a time when in vitro predictive models of metabolic DDI were not an integral part of the lead optimization process. Because of that, its metabolic DDI liabilities were discovered, after the drug had been approved.

Fluvoxamine is metabolized in a stepwise manner by CYP450 system to give 3 metabolites having progressively higher oxidative levels: an O-desmethyl 2 (an alcohol), an aldehyde 3, and finally a carboxylic acid metabolite 4 which is the major metabolite in man. The major metabolite 4 does not undergo any further metabolism and is safely eliminated by renal fillration. This sequence of oxidative events is responsible for DDI and toxicity in man.

By applying the concept of a non-oxidative alternative metabolic pathway, one can design a fluvoxamine analog 5 by introducing a hydrolysable bond into the fluvoxamine structure. Compound 5, like fluvoxamine, binds to the serotonin transporter and has serotonin reuptake inhibition properties similar to fluoxetine in vitro. The major improvement over fluvoxamine is that Compound 5 is metabolized in one step by non-oxidative hydrolytic enzymes to the same major carboxylic acid metabolite 4 as fluvoxamine. This fluvoxamine analog is, therefore, not expected to cause metabolic drug-drug interactions with other drugs that are metabolized by CYP450.

Metabolism-based DDI take place when two (2) or more drugs compete for metabolism by the same enzyme. These metabolic interactions become relevant to DDI when the metabolic system is inducible or/and easily saturable. Such metabolic interactions lead to modification of the pharmacokinetics of the drugs and potential toxicity.

Multiple-drug therapy is a common practice, particularly in patients with several diseases or conditions. Whenever two or more drugs are administered over similar or overlapping time periods, the possibility of drug interactions exists. The ability of a single CYP to metabolize multiple substrates is responsible for the large number of documented clinically significant drug interactions associated with CYP inhibition (Shen, W. W., “Cytochrome P450 monooxygenases and interactions of psychotropic drugs: a five-year update,” Int. J. Psychiatry Med. (1995) 25:277-290; Riesenman, C., “Antidepressant drug interactions and cytochrome P450 system: a critical appraisal,” Pharmacotherapy (1995) 15:84S-99S; Somogyi, A. et al., “Pharmacokinetic interactions of cimetidine,” Clin Pharmacokinet (1987) 12:321-366). The inhibition of drug metabolism by competition for the same enzyme may result in undesirable elevation in plasma drug concentration. In addition, drug interactions can also occur as a result of induction of several CYPs following prolonged drug treatment.

Enzymes of the CYP450 system are ubiquitous oxidative enzymes found in prokaryotes and eukaryotes. They exist as a superfamily of closely related isozymes, whose substrates comprise a wide variety of structurally unrelated compounds. The enzymes can exhibit broad substrate specificity, but a particular substrate may also be metabolized by several different isozymes. CYP450 play a primary role in the metabolism of drugs and xenobiotics.

The clinical significance of a metabolic drug-drug interaction depends on the magnitude of the change in the concentration of active species (parent drug and/or active metabolites) at the site of pharmacological action and the therapeutic index of the drug. Observed changes arising from metabolic drug-drug interactions can be substantial (e.g., an order of magnitude or more decrease or increase in the blood and tissue concentrations of a drug or metabolite) and can include formation of toxic metabolites or increased exposure to a toxic parent compound.

Examples of substantially changed exposure associated with administration of another drug include (1) increased levels of terfenadine, cisapride, or astemizole with ketoconazole or erythromycin (inhibition of CYP3A4); (2) increased levels of simvastatin and its acid metabolite with mibefradil or itraconazole (inhibition of CYP3A4); (3) increased levels of desipramine with fluoxetine, paroxetine, or quinidine (inhibition of CYP2D6); and (4) decreased carbamazepine levels with rifampin (induction of CYP3A4).

These large changes in exposure can alter the safety and efficacy profile of a drug and/or its active metabolites in important ways. This is most obvious and expected for a drug with a narrow therapeutic range (NTR), but is also possible for non-NTR drugs as well (e.g., HMG CoA reductase inhibitors). Patients receiving anticoagulants, antidepressants or cardiovascular drugs are at a much greater risk than other patients because of the narrow therapeutic index of these drugs. Although most metabolic drug-drug interactions that can occur with these agents are manageable, usually by appropriate dosage adjustment, a number of these DDI are potentially life threatening.

As an example, mibefradil (Posicor®), a calcium channel blocker has been used for the management of hypertension and chronic stable angina (Bursztyn, M., et al., “Mibefradil, a novel calcium antagonist, in elderly patients with hypertension: favorable hemodynamics and pharmacokinetics,” Am. Heart J. (1997) 134:238-247). Mibefradil inhibits CYP3A4 and interferes with the metabolism of CYP3A4 substrates. Several clinical trials described the overall safety of mibefradil. However, the populations studied were probably healthier and more closely supervised than what is seen in routine clinical practice. After potentially serious interactions between mibefradil and beta-blockers, digoxin, verapamil, and diltiazem, were reported, mibefradil was voluntarily withdrawn from the market in 1998. Clinicians began the switch from mibefradil to alternative antihypertensive agents, often choosing dihydropyridine-type calcium-channel blockers (CCB), such as nifedipine. A report described four cases of cardiogenic shock in patients taking mibefradil and beta-blockers who were switched to dihydropridine CCBs after withdrawal of mibefradil from the market. One case resulted in death; the other 3 patients survived episodes of cardiogenic shock requiring intensive support of heart rate and blood pressure. All cases occurred within 24 hours of discontinuing mibefradil and initiating the dihydropyridine CCBs. This serious drug-drug interaction probably occurred for two reasons. First, both mibefradil and dihydropyridines are substrates for CYP3A4, making this a potential mechanism. Second, mibefradil has a long half-life (up to 24 hours), with therapeutic levels of the agent likely to have been present within 24 hours of discontinuation.

The development of new chemical entities (NCE) that do not induce or inhibit CYP450 and whose metabolism is not altered by other drugs is highly desirable and are sought by pharmaceutical companies.

An alternate, non-CYP450 metabolic pathway, designed into the drug structure can minimize the chances of CYP450-based drug-drug interactions. In other words, an alternate, non-CYP450, metabolic pathway acts as a built-in escape route when a multi-drug therapeutic regimen causes CYP450 interactions to occur. For example, fenoldopam, an antihypertensive agent, is metabolized via 3 parallel and independent metabolic routes that are not based on CYP450: methylation via catechol O-methyl transferase, glucuronidation, and sulfation. Similarly, raloxifene undergoes extensive first pass metabolism by the liver and the major metabolites are the 6-glucuronide, the 4′-glucuronide, and the 6,4′-diglucuronide conjugates, which are not dependent on CYP450. Consequently, no significant metabolic drug interactions with inhibitors of CYP450 are known for fenoldopam and raloxifene.

Remifentanil, an ultra-short opioid used as analgesic during induction and maintenance of general anesthesia, further illustrates this point. Remifentanyl is metabolized extensively by esterases, which are non-oxidative, not CYP450-dependent, enzymes. Following i.v. administration, remifentanil is rapidly metabolized in the blood and other tissues. As a consequence, the elimination of remifentanil is independent of renal and hepatic function (Dershwitz, M., et al., “Pharmacokinetics and pharmacodynamics of remifentanil in volunteer subjects with severe liver disease,” Anesthesiology (1996) 84:812-820), and no clinically significant metabolic drug-drug interactions have been reported.

Elevation of LFT can be idiosynchratic, i.e., its true source is unknown but is probably linked to a genetic variation in the patient population. However, the vast majority of LFT elevations are not idiosynchratic. Regardless, LFT elevations are a direct indicator of hepatocyte toxicity and are due to the accumulation of a toxic compound in hepatocytes. The term accumulation is used herein to indicate that the concentrations of toxic compound in the hepatocyte is larger than that which can be safely eliminated by the cell. The toxic compound can be either the drug itself or the metabolite(s).

In some cases, LFT elevations can be traced to the formation of a reactive metabolic intermediate. The body has natural detoxification systems to eliminate reactive intermediates. When the detoxification systems fail, reactive intermediates are free to react with endogenous molecules, proteins, and even DNA, thus leading to carcinogenicity, theratogenicity, mutations, etc. A well-known example is the carcinogenicity of benzene due to the formation of a reactive epoxide intermediate. This epoxide is normally detoxified by glutathione and/or an epoxide hydrolase. When amounts of benzene are too high however, epoxide hydrolase and glutathione are saturated, and the epoxide becomes toxic, producing rapid LFT elevations and longer-term carcinogenicity.

In other cases, it is the accumulation of the drug itself or one of its metabolites, into the hepatocytes that are the cause of LFT elevations. An example of this is troglitazone (Rezulin®). In primary human hepatocyte culture there is a strong positive correlation between hepatocyte toxicity and lack of metabolism of troglitazone, resulting in accumulation and cell death (Kostrubsky, V. E., et al., “The role of conjugation in hepatotoxicity of troglitazone in human and porcine hepatocyte cultures,” Drug Metab. Dips. (2000) 28:1192-1197).

Torsade de pointes is a potentially life-threatening cardiac arrhythmia associated with blockade of the rapidly activating component of delayed rectifier potassium channels (IKr) in the myocardium. This channel is expressed from the human homologue of the ether-a-go-go related gene and as such is often referred to by its acronym as the HERG channel (Vandenberg, J. I., et al, “HERG K+ channels: friend and foe,” TIPS (2001) 22:240-6). The arrhythmia resulting from blockade of this receptor is characterized by a dose-dependent prolongation of the QT interval of the surface electrocardiogram. The novel compounds and methods provided by this invention eliminate, or significantly reduce, this undesired activity by optimizing the pharmacology and pharmacodynamics of the metabolite as well as the pharmacokinetics of the drug itself.

QT prolongation resulting in fatal TDP can also be traced to metabolic sources. QT prolongation and TDP happen in the presence of compounds that block the ventricular IK_{R} channel (Herg channel), therefore delaying repolarization of the ventricle and leading to unresponsiveness of the ventricular muscle to further stimulus and depolarization. The blocking activity on the Herg channel is usually concentration-dependent. Thus, a weak Herg-channel blocker that does not reach inhibitory concentrations at normal therapeutic doses is considered safe. However, when circumstances cause blood levels to rise above normal therapeutic levels and reach levels where IK_{R} inhibition is substantial, then a small fraction of the population who are genetically predisposed become suddenly at high risk of developing TDP.

This phenomenon of drug accumulation over time can be caused by several factors. In the simplest case it can be an accidental overdose. In other instances, it can be caused by non-linear pharmacokinetics of the drug. The most common reason however is when blood levels suddenly rise due to DDI. This DDI can be at 2 different levels: competition for a carrier-protein binding site, or competition for a metabolizing enzyme. Overdose and DDI were the primary causes for the toxicity of cisapride, a drug that was banned by the FDA in the spring of 2000 for causing unpredictable TDP in patients. The pharmacology of the HERG channel is complex, but it is clear that reducing the lipophilicity and/or increasing the number of hydrogen bonding sites in a molecule tends to lower channel affinity (Guengerich, F. P., “Role of cytochrome P450 enzymes in drug-drug interactions,” Drug-drug interactions: scientific and regulatory perspectives (1997) 7-35, Li AP (ed.) Academic Press, San Diego). In addition, the drugs of this invention are primarily metabolized by non-oxidative pathways that yield water soluble, polar metabolites. Thus, the primary metabolites have reduced, or are devoid of, affinity for the HERG channel. This feature is exemplified in the discovery of fexofenadine which is a carboxylic acid metabolite of the non-sedating antihistamine terfenadine. Both compounds are active as antihistamines but the relatively lipophilic terfenadine is arrhythmogenic at high plasma levels whereas its metabolite is devoid of such activity (Selnick, H. G., et aL, “Class-III anti-arrhythmic activity in vivo by selective blockade of the slowly activating cardiac delayed rectifier potassium current,” J. Med. Chem. (1997) 40:3865-3868).

The pharmacokinetic profile of a compound is governed by its physicochemical properties. The polarity of a molecule affects its volume of distribution such that polar compounds have a comparatively low volume of distribution. This keeps compounds out of the more lipophilic tissues such as the heart and increases the concentration available in plasma. A comparison between terfenadine and astemizole shows a positive correlation between the volume of distribution and the degree of cardiotoxicity (DePonti, F., et al., “QT-interval prolongation by non-cardiac drugs: lessons to be learned from recent experience,” Eur. J. Clin. Pharmacol. (2000) 56:1-18). A significant proportion of drug-induced episodes of TDP are the result of an unexpected shift in the metabolic pathway due to a drug-drug-interaction, genetic trait, or overdose. The cause is the same in each case: the primary metabolic pathway is blocked and drug accumulates to a toxic level.

The subject invention provides novel compounds and compositions having a metabolic pathway that is well characterized, primarily non-oxidative, and difficult to overwhelm.

### BRIEF SUMMARY

The subject invention provides therapeutically useful and therapeutically effective compounds and compositions for the treatment of a variety of disorders. The compounds of the invention exhibit significantly reduced levels of drug-drug interactions (DDI) and are metabolized, primarily, via non-oxidative systems. Compounds and compositions of the invention are administered to mammals, preferably to humans, for therapeutic purposes.

### DETAILED DISCLOSURE

A drug that has two metabolic pathways, one oxidative and one non-oxidative, built into its structure is highly desirable in the pharmaceutical industry. An alternate, non-oxidative metabolic pathway provides the treated subject with an alternative drug detoxification pathway (an escape route) when one of the oxidative metabolic pathways becomes saturated or non-functional. While a dual metabolic pathway is necessary in order to provide an escape metabolic route, other features are needed to obtain drugs that are safe regarding DDI, TDP, and LFT elevations.

In addition to having two metabolic pathways, the drug should have a rapid metabolic clearance (short metabolic half-life) so that blood levels of unbound drug do not rise to dangerous levels in cases of DDI at the protein level. Also, if the metabolic half-life of the drug is too long, then the CYP450 system again becomes the main elimination pathway, thus defeating the original purpose of the design. In order to avoid high peak concentrations and rapidly declining blood levels when administered, such a drug should also be administered using a delivery system that produces constant and controllable blood levels over time.

The subject invention provides therapeutically useful and effective compounds and compositions for the treatment of a variety of disorders. The compounds of this invention have one or more of the following characteristics or properties:

1. Compounds of the invention are metabolized both by CYP450 and by a non-oxidative metabolic enzyme or system of enzymes;

2. Compounds of the invention have a short (up to four (4) hours) non-oxidative metabolic half-life;

3. Oral bioavailability of the compounds is consistent with oral administration using standard pharmaceutical oral formulations; however, the compounds, and compositions thereof, can also be administered using any delivery system that produces constant and controllable blood levels over time;

4. Compounds according to the invention contain a hydrolysable bond that can be cleaved non-oxidatively by hydrolytic enzymes;

5. Compounds of the invention can be made using standard techniques of small-scale and large-scale chemical synthesis;

6. The primary metabolite(s) of compound(s) of this invention result(s) from the non-oxidative metabolism of the compound(s);

7. The primary metabolite(s), regardless of the solubility properties of the parent drug, is, or are, soluble in water at physiological pH and have, as compared to the parent compound, a significantly reduced pharmacological activity;

8. The primary metabolite(s), regardless of the electrophysiological properties of the parent drug, has, or have, negligible inhibitory activity at the IK_{R} (HERG) channel at normal therapeutic concentration of the parent drug in plasma (e.g., the concentration of the metabolite must be at least five times higher than the normal therapeutic concentration of the parent compound before activity at the IK_{R} channel is observed);

9. Compounds of the invention, as well as the metabolites thereof, do not cause metabolic DDI when co-administered with other drugs;

10. Compounds of the invention, as well as metabolites thereof, do not elevate LFT values when administered alone; and

11. Compounds of the invention are useful for treating a wide range of illnesses, including, but not limited to cardiovascular, metabolic, inflammatory, pain, infections, cancer, gastro-intestinal, mental, pulmonary, urinary, dermatological, and ocular diseases, disorders, or conditions.

In some embodiments, the subject invention provides compounds have any two of the above-identified characteristics or properties. Other embodiments provide for compounds having at least any three of the above-identified properties or characteristics. In another embodiment, the compounds, and compositions thereof, have any combination of at least four of the above-identified characteristics or properties. Another embodiment provides compounds have any combination of five to 10 of the above-identified characteristics or properties. In a preferred embodiment the compounds of the invention have all eleven characteristics or properties.

In various embodiments, the primary metabolite(s) of the inventive compounds, regardless of the electrophysiological properties of the parent drug, has, or have, negligible inhibitory activity at the IK_{R} (HERG) channel at normal therapeutic concentrations of the drug in plasma. In other words, the concentration of the metabolite must be at least five times higher than the normal therapeutic concentration of the parent compound before activity at the IK_{R} channel is observed. Preferably, the concentration of the metabolite must be at least ten times higher than the normal therapeutic concentration of the parent compound before activity at the IK_{R} channel is observed.

Compounds according to the invention are, primarily, metabolized by endogenous hydrolytic enzymes via hydrolysable bonds engineered into their structures. The primary metabolites resulting from this metabolic pathway are water soluble and do not have, or show a reduced incidence of, DDI when administered with other medications (drugs). Non-limiting examples of hydrolysable bonds that can be incorporated into compounds according to the invention include amide, ester, carbonate, phosphate, sulfate, urea, urethane, glycoside, or other bonds that can be cleaved by hydrolases.

Additional modifications of the compounds disclosed herein can readily be made by those skilled in the art. Thus, analogs, derivatives, and salts of the exemplified compounds are within the scope of the subject invention. With a knowledge of the compounds of the subject invention skilled chemists can use known procedures to synthesize these compounds from available substrates. As used in this application, the terms “analogs” and “derivatives” refer to compounds which are substantially the same as another compound but which may have been modified by, for example, adding additional side groups. The terms “analogs” and “derivatives” as used in this application also may refer to compounds which are substantially the same as another compound but which have atomic or molecular substitutions at certain locations in the compound.

The subject invention further provides novel drugs that are dosed via drug delivery systems that achieve slow release of the drug over an extended period of time. These delivery systems maintain constant drug levels in the target tissue or cells. Such drug delivery systems have been described, for example, in Remington: The Science and Practice of Pharmacy, 19^{th} Ed., Mack Publishing Co., Easton, Pa. (1995) pp 1660-1675, which is hereby incorporated by reference in its entirety. Drug delivery systems can take the form of oral dosage forms, parenteral dosage forms, transdermal systems, and targeted delivery systems.

Oral sustained-release dosage forms are commonly based on systems in which the release rate of drug is determined by its diffusion through a water-insoluble polymer. There are basically two types of diffusion devices, namely reservoir devices, in which the drug core is surrounded by a polymeric membrane, and matrix devices, in which dissolved or dispersed drug is distributed uniformly in an inert, polymeric matrix. In actual practice, however, many diffusion devices also rely on some degree of dissolution in order to govern the release rate.

Dissolution systems are based on the fact that drugs with slow dissolution rates inherently produce sustained blood levels. Therefore, it is possible to prepare sustained-release formulations by decreasing the dissolution rate of highly water-soluble drugs. This can be carried out by preparing an appropriate salt or other derivative, by coating the drug with a slowly soluble material, or by incorporating it into a tablet with a slowly soluble carrier.

In actual practice, most of the dissolution systems fall into two categories: encapsulated dissolution systems and matrix dissolution systems. Encapsulated dissolution systems can be prepared either by coating particles or granules of drug with varying thicknesses of slowly soluble polymers or by micro-encapsulation, which can be accomplished by using phase separation, interfacial polymerization, heat fusion, or the solvent evaporation method. The coating materials may be selected from a wide variety of natural and synthetic polymers, depending on the drug to be coated and the release characteristics desired. Matrix dissolution devices are prepared by compressing the drug with a slowly soluble polymer carrier into a tablet form.

In osmotic pressure-controlled drug-delivery systems, osmotic pressure is utilized as the driving force to generate a constant release of drug. Additionally, ion-exchange resins can be used for controlling the rate of release of a drug, which is bound to the resin by prolonged contact of the resin with the drug solution. Drug release from this complex is dependent on the ionic environment within the gastrointestinal tract and the properties of the resin.

Parenteral sustained-release dosage forms most commonly include intramuscular injections, implants for subcutaneous tissues and various body cavities, and transdermal devices. Intramuscular injections can take the form of aqueous solutions of the drug and a thickening agent which increases the viscosity of the medium, resulting in decreased molecular diffusion and localization of the injected volume. In this manner, the absorptive area is reduced and the rate of drug release is controlled. Alternatively, drugs can be complexed either with small molecules such as caffeine or procaine or with macromolecules, e.g., biopolymers such as antibodies and proteins or synthetic polymers, such as methylcellulose or polyvinylpyrrolidone. In the latter case, these formulations frequently take on the form of aqueous suspensions. Drugs which are appreciably lipophilic can be formulated as oil solutions or oil suspensions in which the release rate of the drug is determined by partitioning of the drug into the surrounding aqueous medium. The duration of action obtained from oil suspensions is generally longer than that from oil solutions, because the suspended drug particles must first dissolve in the oil phase before partitioning into the aqueous medium. Water-oil (W/O) emulsions, in which water droplets containing the drug are dispersed uniformly within an external oil phase, can also be used for sustained release. Similar results can be obtained from O/W (reverse) and multiple emulsions.

Implantable devices based on biocompatible polymers allow for both a high degree of control of the duration of drug activity and precision of dosing. In these devices, drug release can be controlled either by diffusion or by activation. In diffusion-type implants, the drug is encapsulated within a compartment that is enclosed by a rate-limiting polymeric membrane. The drug reservoir may contain either drug particles or a dispersion (or a solution) of solid drug in a liquid or a solid-type dispersing medium. The polymeric membrane may be fabricated from a homogeneous or a heterogeneous non-porous polymeric material or a microporous or semi-permeable membrane. The encapsulation of the drug reservoir inside the polymeric membrane may be accomplished by molding, encapsulation, microencapsulation or other techniques. Alternatively, the drug reservoir is formed by the homogeneous dispersion of drug particles throughout a lipophilic or hydrophilic polymer matrix. The dispersion of the drug particles in the polymer matrix may be accomplished by blending the drug with a viscous liquid polymer or a semi-solid polymer at room temperature, followed by crosslinking of the polymer, or by mixing of the drug particles with a melted polymer at an elevated temperature. It can also be fabricated by dissolving the drug particles and/or the polymer in an organic solvent followed by mixing and evaporation of the solvent in a mold at an elevated temperature or under vacuum.

In microreservoir dissolution-controlled drug delivery, the drug reservoir, which is a suspension of drug particles in an aqueous solution of a water-miscible polymer, forms a homogeneous dispersion of a multitude of discrete, unleachable, microscopic drug reservoirs in a polymer matrix. The microdispersion may be generated by using a high-energy dispersing technique. Release of the drug from this type of drug delivery device follows either an interfacial partition or a matrix diffusion-controlled process.

In activation-type implants, the drug is released from the semi-permeable reservoir in solution form at a controlled rate under an osmotic pressure gradient. Implantable drug-delivery devices can also be activated by vapor pressure, magnetic forces, ultrasound, or hydrolysis.

Transdermal systems for the controlled systemic delivery of drugs are based on several technologies. In membrane-moderated systems, the drug reservoir is totally encapsulated in a shallow compartment molded from a drug-impermeable backing and a rate-controlling microporous or non-porous polymeric membrane through which the drug molecules are released. On the external surface of the membrane, a thin layer of drug-compatible, hypoallergenic adhesive polymer may be applied to achieve an intimate contact of the transdermal system with the skin. The rate of drug release from this type of delivery system can be tailored by varying the polymer composition, permeability coefficient or thickness of the rate-limiting membrane and adhesive.

In adhesive diffusion-controlled systems, the drug reservoir is formulated by directly dispersing the drug in an adhesive polymer and then spreading the medicated adhesive, by solvent casting, onto a flat sheet of drug-impermeable backing membrane to form a thin drug reservoir layer. On top of the drug-reservoir layer, layers of non-medicated, rate controlling adhesive polymer of constant thickness are applied to produce an adhesive diffusion-controlled drug-delivery system.

In matrix dispersion systems, the drug reservoir is formed by homogeneously dispersing the drug in a hydrophilic or lipophilic polymer matrix. The medicated polymer is then molded into a disc with a defined surface area and controlled thickness. The disc is then glued to an occlusive baseplate in a compartment fabricated from a drug-impermeable backing. The adhesive polymer is spread along the circumference to form a strip of adhesive rim around the medicated disc. In microreservoir systems, the drug reservoir is formed by first suspending the drug particles in an aqueous solution of a water-soluble polymer and then dispersing homogeneously, in a lipophilic polymer, by high-shear mechanical forces to form a large number of unleachable, microscopic spheres of drug reservoirs. This thermodynamically unstable system is stabilized by crosslinking the polymer in situ, which produces a medicated polymer disk with a constant surface area and a fixed thickness.

Targeted delivery systems include, but are not limited to, colloidal systems such as nanoparticles, microcapsules, nanocapsules, macromolecular complexes, polymeric beads, microspheres, and liposomes. Targeted delivery systems can also include resealed erythrocytes and other immunologically-based systems. The latter may include drug/antibody complexes, antibody-targeted enzymatically-activated prodrug systems, and drugs linked covalently to antibodies.

The invention also provides methods of producing these compounds.

It is another aspect of this invention to provide protocols by which these conditions can be tested. These protocols include in vitro and in vivo tests that have been designed to: 1) ensure that the novel compound is metabolized both by CYP450 and by hydrolytic enzymes; 2) that the non-oxidative half-life of the parent drug is no more than a certain value when compared to an internal standard (in preferred embodiments, less than about four hours); 3) that the primary metabolite of the parent drug is the result of non-oxidative metabolism; 4) that the primary metabolite of the parent drug (regardless of the solubility properties of the parent drug) is water soluble; 5) that the primary metabolite of the parent drug (regardless of the electrophysiological properties of the parent drug) has negligible inhibitory properties toward IK_{R} channel at concentrations similar to therapeutic concentration of the parent drug; 6) that the novel compound (regardless of its properties) does not cause metabolic DDI when co-administered with other drugs; and 7) that the novel compound does not cause hepatic toxicity in primary human hepatocytes.

### EXAMPLE 1

CYP Assays

A series of assays to test for activity of 5 principal drug metabolizing enzymes, CYP1A4, CYP2C9, CYP2C19, CYP2D6, and CYP3A4, as well as other CYP450 subfamil have been designed and are now commercially available either as ready-to-use kits or as contract work. Commercial sources for these assays include for example Gentest and MDS Panlabs. These assays can test for activity of the enzyme toward metabolism of the test compound as well as testing for kinetic modification (inhibition or activation) of the enzyme by the substrate. These in vitro protocols use simple rapid, low cost methods to characterize aspects of drug metabolism and typically require less than 1 mg of test material.

### EXAMPLE 2

High Throughput Cytochrome P450 Inhibition Screen

The majority of drug-drug interactions are metabolism-based and of these, most involve CYP450. For example, if a new chemical entity is a potent CYP450 inhibitor, it may inhibit the metabolism of a co-administered medication, potentially leading to adverse clinical events. The inhibition of human CYP1A2, CYP2C8, CYP2C9, CYP2C19, CYP2D6, CYP3and other isoforms are assessed using microsomal preparations as enzyme sources and the fluorescence detection method described in the literature (Crespi, C. L., et al., “Microtiter plate assays for inhibition of human, drug-metabolizing cytochromes P450,” Anal. Biochem. (1997) 248:188-190; Crespi, C. L., et al., “Novel High throughput fluorescent cytochrome P450 assays,” Toxicol. Sci. (1999) 48, abstr. No.323; Favreau, L. V., et al., “Improved Reliability of the Rapid Microtiter Plate Assay Using Recombinant Enzyme in Predicting CYP2D6 Inhibition in Human Liver Microsomes,” Drug Metab. Dispos. (1999) 27:436-439). Tests are conducted in 96-well microtiter plates and may use the following fluorescent CYP450 substrates: resorufin benzyl ether (BzRes), 3-cyano-7-ethoxycoumarin (CEC), ethoxyresorufin (ER), 7-methoxy-4-trifluoromethylcoumarin (MFC), 3-[2-(N,N-diethyl-N-methylamino)ethyl]-7-methoxy-4-methylcoumarin (AMMC), 7-benzyloxyquinoline (BQ), dibenzyfluorescein (DBF) or 7-benzyloxy-4-trifluoromethylcoumarin (BFC). Multiple CYP3A4 substrates are available to assess substrate dependence of IC₅₀ values, activation and the complex inhibition kinetics associated with this enzyme (Korzekwa, K. R., et al., “Evaluation of atypical cytochrome P450 kinetics with two-substrate models: evidence that multiple substrates can simultaneously bind to the cytochrome P450 active sites,” Biochemistry (1998) 37:4137-47; Crespi, C. L., “Higher-throughput screening with human cytochromes P450,” Curr. Op. Drug Discov. Dev. (1999) 2:15-19). Data are reported as IC₅₀ values or percent inhibition when using only one or two concentrations of test compound.

### EXAMPLE 3

Metabolic Stability

Metabolic stability influences both oral bioavailability and half-life; compounds of higher metabolic stability are less controllable in their pharmacokinetic parameters. This combination of characteristics, or properties, leads to potential DDI and liver toxicity. This test measures the metabolic stability of the compound in the presence of CYP450, in the presence of hydrolytic enzymes, and in the presence of both CYP450 and hydrolytic enzymes.

Stability in the presence of CYP450: With CYP450 substrates of low and moderate in vivo clearance, there is a good correlation between in vitro metabolic stability and in vivo clearance (Houston, J. B., “Utility of in vitro drug metabolism data in predicting in vivo metabolic clearance,” Biochem Pharmacol. (1994) 47(9):1469-7). This test uses pooled liver microsomes, S9 (human and/or preclinical species) or microsomal preparations with appropriate positive and negative controls. Assessment of both phase-I and phase-II enzymatic metabolism is possible, and a standard set of substrate concentrations and incubations may be used. Metabolism is measured by loss of parent compound HPLC analysis with absorbance, fluorescence, radiometric or mass spectrometric detection can be used.

Stability in the presence of hydrolytic enzymes: Hydrolytic enzymes in liver cytosol, plasma, or enzymatic mixes from commercial sources (human and/or preclinical species) are used to assess the metabolic stability of the novel compounds of the invention. Appropriate positive and negative controls as well as a standard set of substrate concentrations are added in order to correlate in vitro observations with in vivo metabolic half-life. Metabolism is measured by loss of parent compound. HPLC analysis with absorbance, fluorescence, radiometric or mass spectrometric detection can also be used.

Stability in the presence of both CYP450 and hydrolytic enzymes: This test uses pooled liver microsomes, S9 (human and/or preclinical species) or microsomal preparations with appropriate positive and negative controls, combined with hydrolytic enzymes from commercial sources, plasma, or cytosol to assess metabolic stability. The test can also be performed in primary hepatocytes (human and/or preclinical species) or in perfused liver (preclinical species). The use of positive and negative controls, as well as a standard set of substrates allow for correlations between in vitro observations and in vivo metabolic half-life.

### EXAMPLE 4

CYP1A1 Induction Screening

Induction of CYP1A1 is indicative of ligand activation of the aryl hydrocarbon (Ah) receptor, a process associated with induction of a variety of phase-I and phase-II enzymes (Swanson, H. I., “The AH-receptor: genetics, structure and function,” Pharmacogenetics (1993) 3:213-30). Many pharmaceutical companies choose to avoid development of compounds suspected as Ah-receptor ligands. This test uses a human lymphoblastoid cell line containing native CYP1A1 activity that is elevated by exposure to Ah receptor ligands. Assays are conducted in 96-well microtiter plates using an overnight incubation with the test substances, followed by addition of 7-ethoxy-4-trifluoromethylcoumarin as substrate. Dibenz(a,h)anthracene is used as a positive control inducer. A concurrent control test for toxicity or CYP1A1 inhibition is available using another cell line that constitutively expresses CYP1A1.

### EXAMPLE 5

Cytochrome P450 Reaction Phenotyping

The number and identity of CYP450 enzymes responsible for the metabolism of a drug affects population variability in metabolism. Reaction phenotyping uses either liver microsomes with selective inhibitors or a panel of cDNA-expressed enzymes to provide a preliminary indication of the number and identity of enzymes involved in the metabolism of the substrate. The amount of each cDNA-expressed enzyme is chosen to be proportional to the activity of the same enzyme in pooled human liver microsomes. Protein concentration is standardized by the addition of control microsomes (without CYP450 enzymes). A standard set of substrate concentrations and incubations is used and metabolism of the drug is measured by loss of parent compound. Alternatively, HPLC analysis with absorbance, fluorescence, radiometric or mass spectrometric detection can be used.

### EXAMPLE 6

Drug Permeability Measurement in Caco-2, LLC-PK1 or MDCK Cell Monolayers

Drug permeability through cell monolayers correlates well with intestinal permeability and oral bioavailability. Several mammalian cell lines are appropriate for this measurement (Stewart, B. H., et al., “Comparison of intestinal permeabilities determined in multiple in vitro and in situ models: relationship to absorption in humans,” Pharm. Res. (1995) 12:693-9; Irvine, J. D., et al., “MDCK (Madin-Darby Canine Kidney) cells: A tool for membrane permeability screening,” J. Pharm. Sci. (1999) 88:28-33). Apical to basolateral diffusion is measured using a standard set of time points and drug concentrations. These systems can be adapted to a high throughput mode. Liquid chromatography/mass spectroscopy (LC/MS) analysis is also available for analysis of metabolites. Controls for membrane integrity and comparator compounds are included and data are reported as apparent permeability (Pₐₚₚ) or percent flux under fixed conditions.

### EXAMPLE 7

Human P-glycoprotein (PGP) Screen

An ATPase assay is used to determine if the compounds interact with the xenobiotic transporter MDR1 (PGP). ATP hydrolysis is required for drug efflux by PGP, and the ATPase assay measures the phosphate liberated from drug-stimulated ATP hydrolysis in human PGP membranes. The assay screens compounds in a high throughput mode using single concentration determinations compared to the ATPase activity of a known PGP substrate. A more detailed approach by determining the concentration-dependence and apparent kinetic parameters of the drug-stimulated ATPase activity, or inhibitory interaction with PGP can also be used.

### EXAMPLE 8

PGP-Mediated Drug Transport in Polarized Cell Monolayers

P-glycoprotein (PGP) is a member of the ABC transporter superfamily and is expressed in the human intestine, liver and other tissues. Localized to the cell membrane, PGP functions as an ATP-dependent efflux pump, capable of transporting many structurally unrelated xenobiotics out of cells. Intestinal expression of PGP may affect the oral bioavailability of drug molecules that are substrates for this transporter. Compounds that are PGP substrates can be identified by direct measurement of their transport across polarized cell monolayers. Two-directional drug transport (apical to basolateral permeability, and basolateral to apical PGP-facilitated efflux) can be measured in LLC-PK1 cells (expressing human PGP cDNA) and in corresponding control cells. Caco-2 cells can also be used. Concentration-dependence is analyzed for saturation of PGP-mediated transport, and apparent kinetic parameters are calculated. Test compounds can also be screened in a higher throughput mode using this model. LC/MS analysis is available. Controls for membrane integrity and comparator compounds are included in the assay system.

### EXAMPLE 9

Protein Binding

LC/MS analysis can be used to assess the affinity of the test compound for immobilized human serum albuminn (Tiller, P. R., et al., “Immobilized human serum albumin: Liquid chromatography/mass spectrometry as a method of determining drug-protein binding,” Rapid comm. mass spectrom. (1995) 9:261-3). Appropriate low, medium and high binding positive control comparators are included in the test.

### EXAMPLE 10

Metabolite Production

Milligram quantities of metabolites can be produced using microsomal preparations or cell lines. These metabolites can be used as analytical standards, an aid in structural characterization, or as material for toxicity and efficacy testing.

### EXAMPLE 11

Effect on Herg Channel

This assay tests the effect of parent drugs and metabolite(s) on Herg channels using either a cloned Herg channel expressed in stable human embryonic kidney cells (HEK), or Chinese hamster ovary cells (CHO) transiently expressing the Herg/MiRP-1-encoded potassium channel. Whole cell experiments are carried out by means of the patch-clamp technique and performed in the voltage-clamp mode.

In the test using HEK cells, cells are depolarized from the holding potential of −80 mV to voltages between −80 and +60 mV in 10 mV increments for 4 seconds in order to fully open and inactivate the channels. The voltage is then stepped back to −50 mV for 6 seconds in order to record the tail current. The current is also recorded in the presence of test compounds in order to evaluate a dose-response curve of the ability of a test compound to inhibit the Herg channel.

In the test involving CHO cells, the cells are clamped at a holding potential of −60 mV in order to establish the whole-cell configuration. The cells are then depolarized to +40 mV for 1 second and afterwards hyper-/depolarized to potentials between −120 and +20 mV in 20 mV increments for 300 mSec in order to analyze the tail currents. To investigate the effects of test compounds, the cells are depolarized for 300 mSec to +40 mV and then repolarized to −60 mV at a rate of 0.5 mV/mSec, followed by a 200-mSec test potential to -−120 mV. After 6 control stimulations, the extracellular solution is changed to a solution containing the test compound, and 44 additional stimulations are then performed. The peaks of the outwards currents and inward tail currents are analyzed.

Activity on HERG channel can also be assessed using a perfused heart preparation, usually guinea pig heart or other small animal. In this assay the heart is paced and perfused with a solution containing a known concentration of the drug. A concentration-response curve of the effects of drug on QT interval is then recorded and compared to a blank preparation in which the perfusate does not contain the drug.

### EXAMPLE 12

Toxicity in Hepatocyte Cell Culture

This test is performed in primary human and porcine hepatocyte cultures. Toxicity is determined by the measurement of total protein synthesis by pulse-labeling with [¹⁴C]leucine (Kostrubsky, V. E., et al., “Effect of taxol on cytochrome P450 3A and acetaminophen toxicity in cultured rat hepatocytes: Comparison to dexamethasone,” Toxicol. Appl. Pharmacol. (1997) 142:79-86) and by reduction of 3-(4,5-dimethylthiazol-2-yl)-2,5-diphenyltetrazolium bromide using a protocol described by the manufacturer (Sigma Chemical Co., St. Louis, Mo.). Hepatocytes can be isolated from livers not used for whole organ transplants or from male Hanford miniature pigs.

1. A compound comprising a hydrolysable bond wherein said compound has a combination of three or more of the following characteristics or properties:
a) metabolized both by CYP450 and by a non-oxidative metabolic enzyme or system of enzymes;
b) a non-oxidative metabolic half-life of less than about four hours;
c) oral bioavailability consistent with oral administration using standard pharmaceutical oral formulations of a parent compound;
d) made using standard techniques of small-scale and large-scale chemical synthesis;
e) the primary metabolite(s) of said compound results from the non-oxidative metabolism of the compound;
f) the primary metabolite(s), is, or are, soluble in water at physiological pH and have, as compared to the parent compound, a significantly reduced pharmacological activity;
g) the primary metabolite(s), regardless of the electrophysiological properties of a parent compound, has, or have, negligible inhibitory activity at the IK_{R} (HERG) channel at normal therapeutic concentration of the parent compound in plasma
h) the compound, and metabolite(s) thereof, do not cause metabolic drug-drug interactions (DDI) when co-administered with other drugs; or
i) the compound, and metabolite(s) thereof, do not elevate liver function test (LFT) values when administered alone.

2. The compound according to claim 1, wherein said compound has four or more of the characteristics or properties.

3. The compound according to claim 1, wherein said compound has five or more of the characteristics or properties.

4. The compound according to claim 1, wherein said compound has six or more of the characteristics or properties.

5. The compound according to claim 1, wherein said compound has seven or more of the characteristics or properties.

6. The compound according to claim 1, wherein said compound has eight or more of the characteristics or properties.

7. The compound according to claim 1, wherein said compound has all nine of the characteristics or properties.

8. The compound according to claim 1, wherein said compound has at least the following characteristics or properties: 1a), 1b), and 1e); 1a), 1b), and 1f); 1a), 1b) and 1g); 1a), 1b), and 1h); 1a), 1b), and 1i); 1a), 1e), and 1f); 1a), 1e), and 1g); 1a), 1e), and 1h); 1a), 1e), and 1i); 1a), 1f), and 1g); 1a), 1f), and 1h); 1a), 1f), and 1i); 1a), 1g), and 1h); 1a), 1g), and 1i); 1a), 1h), and 1i); 1b), 1e), and 1f); 1b), 1e), and 1g); 1b), 1e), and = 1h); 1b), 1e), and 1i); 1b), 1f), and 1g); 1b), 1f), and 1h); 1b), 1f), and 1i); 1b), 1g), and 1h); 1b), 1g), and 1i); 1b), 1h), and 1i); 1e), 1f), and 1g); 1e), 1f), and 1h); 1e), 1f), and 1i); 1e), 1g), and 1h); 1e), 1g), and 1i); 1e), 1h), and 1i); 1f), 1g), and 1h); 1f), 1g), and 1i); 1f), 1h), and 1i); or 1g), 1h), and 1i).
